(19) United States
(12) Patent Application Publication
(10) Pub. No.: US 2025/0041850 A1
(43) Pub. Date: Feb. 6, 2025

(54) Potassium sensor membrane composition

(71) Applicant: Proton Intelligence Inc., Gibsons (CA)

(72) Inventors: Sahan Ranamukhaarachchi, Narre Warren (AU); Victor Javier Cadarso Busto, Mentone (AU); Esma Dervisevic, Clayton South (AU); Amane Shiohara, Armadale (AU); Ryan E. Anderson, Dillon, CO (US)

(73) Assignee: Proton Intelligence Inc., Gibsons, BC (CA)

(21) Appl. No.: 18/717,024

(22) PCT Filed: Dec. 15, 2022
(86) PCT No.: PCT/US2022/052927

§ 371 Date: Jun. 6, 2024

Related U.S. Application Data
(60) Provisional application No. 63/291,804, filed on Dec. 20, 2021.

(51) Int. Cl. B01L 3/00

(52) U.S. Cl. CPC B01L 3/502715; B01L 2300/0645; B01L 2300/0681

(57) ABSTRACT

The present invention provides a K⁺ selective membrane compositions for use with K⁺ selective sensors. The composition contains the K⁺ ionophore KI-II, the cation exchanger potassium tetrakis(4-chlorophenyl) borate), the base membrane polymer polyurethane, and the plasticiser dioctyl sebacate. These components are present in the amounts of K⁺ ionophore (KI-II): 15 wt % (ionophore II); Cation exchanger (CE): 2.25 wt % (potassium tetrakis(4-chlorophenyl) borate); Plasticiser (DOS): 29.5 wt % (Bis(2-ethylhexyl) sebacate); and Polymer (PU): 58 wt % (polyurethane).

### CROSS-REFERENCE TO RELATED APPLICATIONS

This application claims priority to and is a non-provisional application of U.S. Prov. App. Ser. No. 63/291,804 filed on Dec. 20, 2021.

### BACKGROUND

Potassium (K⁺) selective membranes compositions are known in the art and can include components such as: a K⁺ ionophore (type I) such as valinomycin; a cation exchanger (CE) such as potassium tetrakis(pentafluorophenyl) borate) or potassium tetrakis(4-chlorophenyl) borate); a plasticiser such as bis(2-ethylhexyl) sebacate; and polymer such as poly (vinyl chloride).

A challenge with membrane compositions of the art is that they do not yield sufficient selectivity to be used for K⁺ monitoring where sodium ion (Na) concentration changes may present much higher changes in concentration (an order of magnitude higher) than K⁺, such as 1 mM K⁺ concentration change against a 50 mM Nat concentration change present much higher changes in concentration (an order of magnitude higher) than K⁺, such as 1 mM K⁺ in solution ranges that can be observed for example in blood or interstitial fluid). During testing, a low concentration solution of for example 10 mM Na⁺ is typically employed. Even under such conditions, a change in the sensor response can be observed due to fluctuations in sodium concentration.

Improvements in K⁺ selective membrane compositions for use in K⁺ selective sensor is desired to improve upon sensor response and performance.

### BRIEF SUMMARY OF THE INVENTION

The present invention provides K⁺ selective membrane compositions for use with K⁺ selective sensors which show superior sensor response and performance. The present invention also provides sensors comprising said compositions and methods of use of said compositions and sensors.

In a first embodiment, the present invention provides a first functional K⁺ sensor membrane composition for use with a K⁺ specific sensor. The membrane composition comprising, consisting of, or consisting essentially of: a K⁺ ionophore comprising (KI-II), a cation exchanger comprising (potassium tetrakis(4-chlorophenyl) borate), base membrane polymer (polyurethane), and a plasticiser (dioctyl sebacate). The composition has a wt %, mol % ratio of KI-II/CE of: 0-30.0.

In a second embodiment, the present invention provides another functional K⁺ sensor membrane composition for use with a K⁺ specific sensor. The membrane composition comprising, consisting of, or consisting essentially of: a K⁺ ionophore comprising (KI-II), a cation exchanger comprising (potassium tetrakis(4-chlorophenyl) borate), a base membrane polymer (such as polyurethane), and a plasticiser (such as dioctyl sebacate). The composition has a wt % ratio base membrane polymer/plasticizer of: 0-10 (e.g. 0/10-10/1 wt %).

In a third embodiment, the present invention provides a functional K⁺ sensor membrane composition for use with a K⁺ specific sensor. The composition comprising, consisting of, or consisting essentially of: a K⁺ ionophore comprising KI-II, a cation exchanger comprising potassium tetrakis(4-chlorophenyl) borate, base membrane polymer comprising polyurethane, and plasticiser (dioctyl sebacate (DOS)). The components are present in the following amounts: K⁺ ionophore (KI-II): 15 wt % (ionophore II); Cation exchanger (CE): 2.25 wt % (potassium tetrakis(4-chlorophenyl) borate); Plasticiser (DOS): 29.5 wt % (Bis(2-ethylhexyl) sebacate); and Polymer (PU): 58 wt % (polyurethane).

### BRIEF DESCRIPTION OF THE FIGURES

FIG. 1 shows the sensor response results from the example section.

FIG. 2 shows sensor calibration plots from the example section.

FIGS. 3 and 4 show sensor response results from the example section.

FIG. 5 shows an EMF curve from the example section.

FIG. 6 shows electrodeposition of PPy using chronoamperometry as described in the example section.

FIG. 7 shows preliminary optimization studies as described in the example section.

FIG. 8 shows preliminary cytotoxicity studies as described in the example section.

### DETAILED DESCRIPTION OF THE INVENTION

The present invention provides a K⁺ selective membrane compositions for use with K⁺ selective sensors. The present Inventors have unexpectedly found that use of such membrane compositions with K⁺ selective sensors, shows superior sensor response and performance.

In a first embodiment, the present invention provides a functional K⁺ sensor membrane composition for use with a K⁺ specific sensor. The membrane composition comprises, consists of, or consists essentially of: a K⁺ ionophore (K), a cation exchanger (CE, a base membrane polymer, and a plasticiser. The composition has a wt %, mol % ratio of K/CE of: 0-30.0 wt %, 0-20.6 mol % (e.g. 0/10-30/1 wt %;); more preferably 0.6-10.0 wt %, 0.97-14.5 mol %, (e.g. 2/3-30/3 wt %); and most preferably 3.3-6.7 wt %, 4.8-9.7 mol %, (e.g. 10/3-10/1.5 wt %).

In a further embodiment, either alone or in combination with any composition described herein, the membrane composition includes: a K⁺ ionophore (K), a cation exchanger (CE), a base membrane polymer (BMP), and a plasticiser (P). In this embodiment, the composition has a wt % ratio BMP/P wt % of: 0-10 (e.g. 0/10-10/1 wt %); more preferably 0.2-4 (e.g. 1/5-4/1 wt %); and most preferably 0.3-2.5 (e.g. 1/3-5/2 wt %).

In yet another preferred embodiment, either alone or in combination with any composition herein described, the membrane composition includes a K⁺ ionophore (K), a cation exchanger (CE), a base membrane polymer (BMP), and a plasticiser (P). In this embodiment, the components are present in the following amounts: K—15 wt %; CE:−2.25 wt %; P—29.5 wt % (Bis(2-ethylhexyl) sebacate); and BMP—58 wt %.

The components K, CE, BMP, and P are not particularly limited herein. However it is noted that in particularly preferred embodiments, the components include any or all of the following.

The K⁺ ionophore is not particularly limited herein. The word ionophore is derived from Greek (ion and phore, “ion carrier”) and is a chemical species that reversibly binds ions and is capable of transporting ions across membranes. Synthetic potassium ionophores include (I=valinomycin, II, III, IV) and all can potentially be employed in a hydrophobic membrane. The most commonly used K⁺ ionophores are ranked I>II>III, and the least common is type IV.

Biologically derived molecules that act as ionophores also exist and can be employed which include other biological functions such as antimicrobial, anti-inflammatory, and antibiotic functions etc. These biologically derived ionophores can be produced using Streptomyces. Examples of such ionophores include: 1) Salinomycin has a high preference to K+, in addition to other alkali metal ions such as Na+, Ca2+ and Mg2+; 2) Bafilomycin A1; 3) Nagericin, reactive with both K+ and H+ ions; 4) Nonactin, reactive with both K+ and NH4+; and 5) Gramicidins, type D is formed from type A, B, and C. Gramicidins form transport channels in the cell membrane through which K+ and Na+ can pass and can be derived from Bacillus.

In preferred embodiments, however, the K⁺ ionophore preferably comprises, consists of, or consists essentially of potassium ionophore II ((KI-II) (CAS: 69271-98-3) which is also known as bis[(benzo-15-crown-5)-15-ylmethyl] pimelate or bis(2,5,8,11,14-pentaoxabicyclo[13.4.0]nonadeca-1 (15), 16,18-trien-17-ylmethyl) heptanedioate and has the structure:

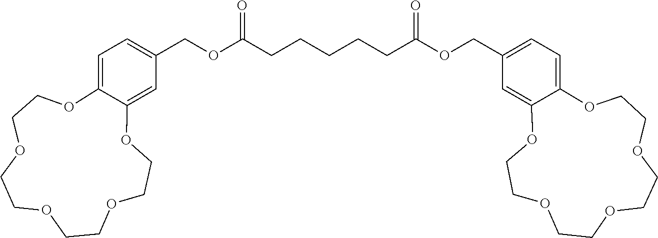 [SMILES: O=C(CCCCCC(=O)OCc1ccc2c(c1)OCCOCCOCCOCCO2)OCc1ccc2c(c1)OCCOCCOCCOCCO2]

The cation exchanger (CE) is not particularly limited and can include for example lipophilic salts such as tetraphenylborate-based lipophilic salts. In preferred embodiments, the CE preferably comprises, consists of, or consists essentially of potassium tetrakis(4-chlorophenyl) borate), which has the following structure:

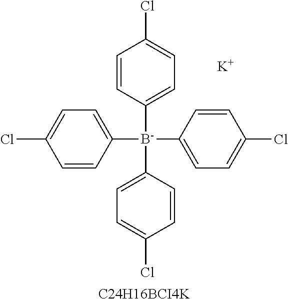 [SMILES: Clc1ccc([B-](c2ccc(Cl)cc2)(c2ccc(Cl)cc2)c2ccc(Cl)cc2)cc1 ; [K+] ; "C24H16BCI4K"]

The plasticizer is not particularly limited but preferably comprises, consists of, or consists essentially of a compound or compounds selected from the group consisting of succinic acid derived plasticizers and/or compounds having a long hydrocarbon chain with nitrophenyl ether group (e.g. 2-nitrophenil octyl ether, dodecyl 2-nictrophenyl ether), phthalate derivatives (e.g. dibutyl phthalate, dioctyl phthalate), and bis(1-butylpentyl) decane-1,10-diyl diglutarate. In most preferred embodiments, the plasticizer comprises, consists of, or consists essentially of Dioctyl sebacate (di(2-ethylhexyl) sebacate) (DOS). DOS is an oily colorless liquid and is an organic compound which is the diester of sebacic acid and 2-ethylhexanol. DOS has the following structure:

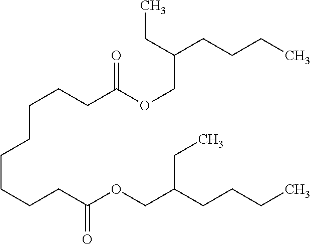 [SMILES: CCCCC(CC)COC(=O)CCCCCCCCC(=O)OCC(CC)CCCC]

The base membrane polymer preferably comprises, consists of, or consists essentially of a polymer selected from the group consisting of: polyurethanes (PU), polydimethylsiloxane (PDMS), polyvinyl chloride (PVC), polyether ether ketone (PEEK), cycloolefin copolymer (COC), polymethyl methacrylate (PMMA), polystyrene, acrylates, poly(vinyl butyral), polyamide, polyimide, and Teflon. In most preferred embodiments the polymer comprises, consists of, or consists essentially of PU. In preferred embodiments PU has a molecular weight of 100 kg/mol and has the following structure:

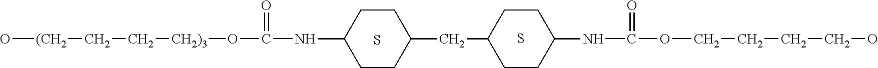

In a most preferred embodiment, the present inventors have discovered that the membrane concentration (ratio) recipe of: 15/2.25, KI-II/CE wt % ratio, and 2/1, PU/DOS wt % ratio unexpectedly produces optimum selectivity to K⁺, particularly when tested with 2 mM LiCl, 2 mM MgCl₂, 4 mM CaCl₂, and 50 mM NaCl in artificial ISF solution.

The membrane compositions may contain additional components added in amounts such that the ratios described above are maintained. These additional components can be added to perform functions that are known in the art. For example, additional components can include materials such as conductive materials including nanomaterials or pyrrole type of monomers that do not significantly affect the membrane performance and selectivity yet might increase the sensitivity.

Additional components can also include redox markers introduced to the membrane in order to enable Voltammetry-based (e.g. DPV, SWV, and CV) detection of the ions. These redox markers are well-known in the art and are described inter alia at: https://analyticalsciencejournals.onlinelibrary.wiley.com/doi/10.1002/elan.201800080).

Other additives (e.g. additional components) might be also introduced to modify mechanical or surface chemistry properties of the membrane, such as to increase adhesion, modify the hydrophobicity/hydrophilicity, tune stiffness or enable enhanced anti-biofouling properties.

The K⁺ sensor membrane compositions as described herein are particularly suitable for use in biological and biomedical applications (for example in connection with potassium sensors for determining potassium in biological fluids, such as blood and/or interstitial fluid). This is due to the biocompatibility of all membrane components. As demonstrated in the example section, preferred membranes showed no significant difference in cell viability after 24 hrs exposure with direct contact. The p values of the preferred membranes with and without PPy were 0.11 and 0.12 respectively which are above the standard cut off value of 0.05 for biocompatibility purposes. No significant morphological difference is observed between a negative control and both of the preferred membrane compositions. Accordingly, in another embodiment, the present invention provides a method of use of the herein described membrane compositions, alone or in combination with a sensor, in vivo and/or in biomedical applications and/or in biomedical apparatuses. In another embodiment, the present membrane compositions can be used in connection with a potassium sensor in biological samples such as blood and/or interstitial fluid and are useful for determining potassium levels in a range (e.g. a linear range) of 0.5 to 10 mM K⁺ which cover critically low and high, and normal, blood K⁺ levels. Blood K⁺ levels correlatable with interstitial fluid (ISF) levels.

The present inventors have discovered that the role and amounts of the specific components affect sensor response and performance in unexpected ways. Conventionally, plasticizer is added to the base membrane polymer (usually PVC) to decrease stiffness and provide flexibility thereby decreasing fragility of the resulting sensor and membrane. The present inventors have found however that lower amounts of plasticizer can be used in the present formulations (and specifically when the base polymer is PU). In these embodiments, the present inventors have likewise unexpectedly found that use of lower amounts plasticizer allows for creation of a durable, robust, and flexible membrane and sensor (e.g. still provides cross-linking and plastication of the polymer (e.g. PU)) while at the same time creating a lipophilic environment for the cation exchanger and the ionophore, which is essential for their functionality. The preferred ratios are described above and the total amount of plasticizer (e.g. DOS) employed is significantly less than that reported in the art (less than 1/1, PU/DOS ratio) and does not oversaturate the membrane (reduce likelihood of leakage of DOS) when employed in these amounts.

The Inventors further note that ratio of KI/CE employed in compositions of the art is typically 2/1 in compositions having a reported maximum of 5 wt % KI-I. Without being bound by a particular mechanism of action, it is believed that this ratio is elevated because of the total added amount of CE, which is important for ion transfer from the aqueous solution into the membrane. On the other hand, a total of 5 wt % KI concentration produces membranes that are not selective enough for use in biomedical applications, where the major interferant sodium ion (Na) can reach concentrations of up to 125 mM while the concentration of the analyte of interest (e.g. potassium (K⁺)) is as low as 5 mM.

Without being bound by a particular mechanism of action, the present inventors have unexpectedly discovered that increasing KI concentration results in higher sensitivity and selectivity. It was likewise unexpectedly found that the total CE amount was sufficient at concentrations as low as 2.25 wt % to produce good sensitivity and high selectivity when having 15 wt % KI-II. It was further unexpectedly found that the ratio can be specific to the ionophore molecule that affects the function and selectivity KI-II is significantly less widely used than KI-I (valinomycin) in the K⁺ selective membrane preparations. Without being bound to a specific mechanism of action it is herein believed that: increased ionophore concentrations slow down the sensor response and require increased CE concentration, which compromises the membrane selectivity because of the reactivity of the CE with the majority of monovalent cations; and increased ionophore concentration requires lowering the overall polymer amount in the membrane and this can lead to losing membrane structural integrity.

Reference throughout the specification to “one embodiment,” “another embodiment,” “an embodiment,” “some embodiments,” and so forth, means that a particular element (e.g., feature, structure, property, and/or characteristic) described in connection with the embodiment is included in at least one embodiment described herein, and may or may not be present in other embodiments. In addition, it is to be understood that embodiment and/or the described element(s) may be combined in any suitable manner.

Numerical values in the specification and claims of this application reflect average values. Furthermore, unless indicated to the contrary, the numerical values should be understood to include numerical values which are the same when reduced to the same number of significant figures and numerical values which differ from the stated value by less than the experimental error of conventional measurement technique of the type described in the present application to determine the value.

### EXAMPLES

The present invention will now be further described with respect to the following non-limiting examples:

### Experimental Conditions and Materials

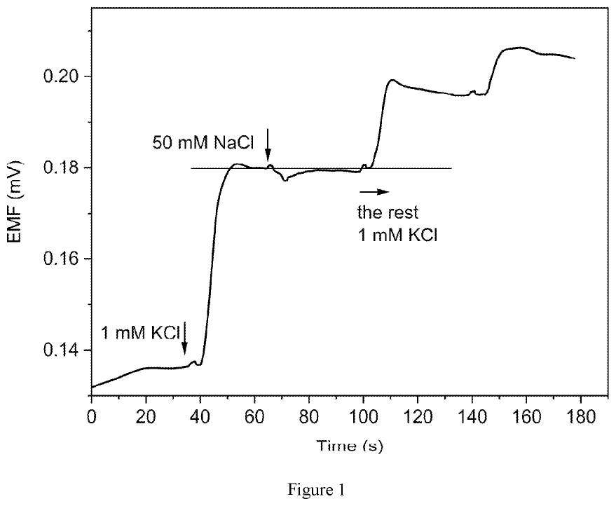

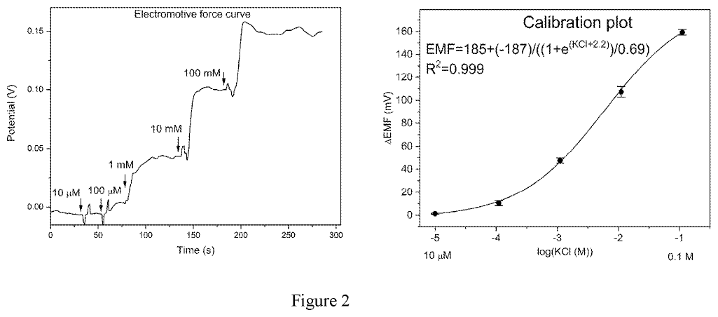

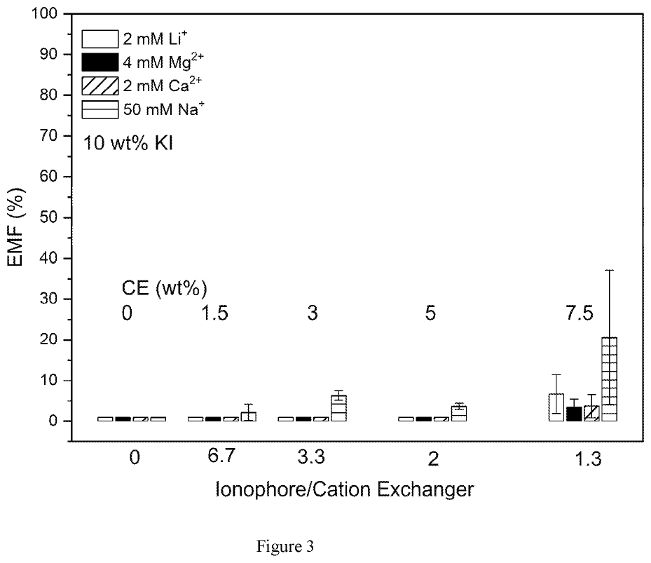

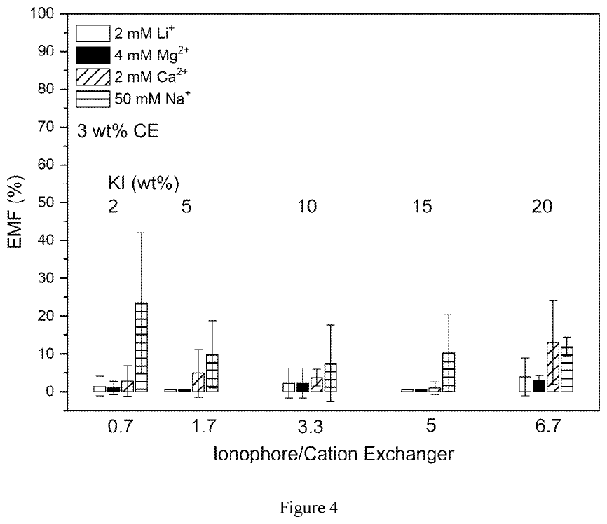

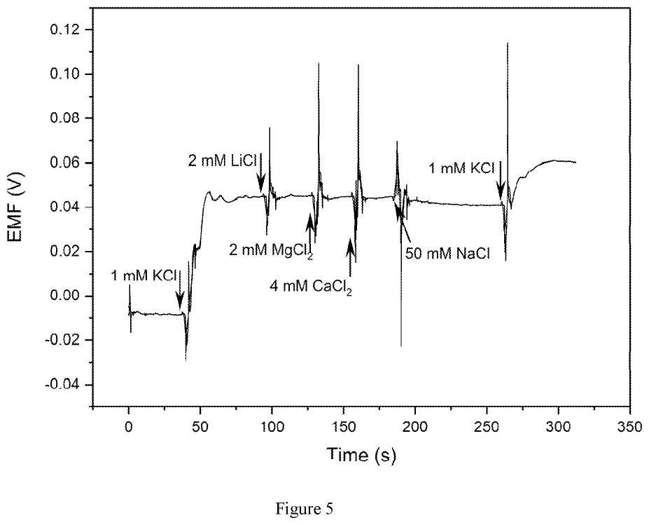

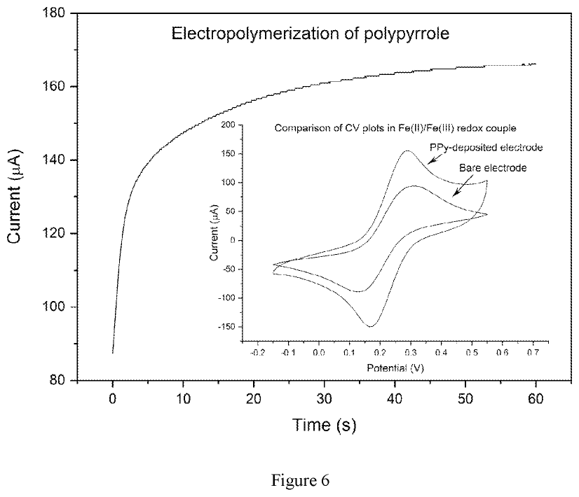

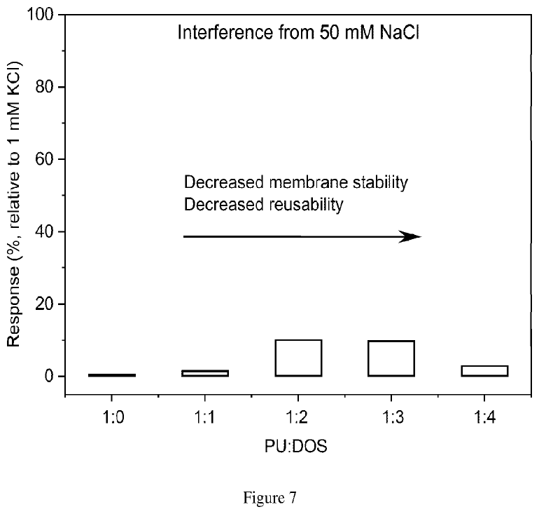

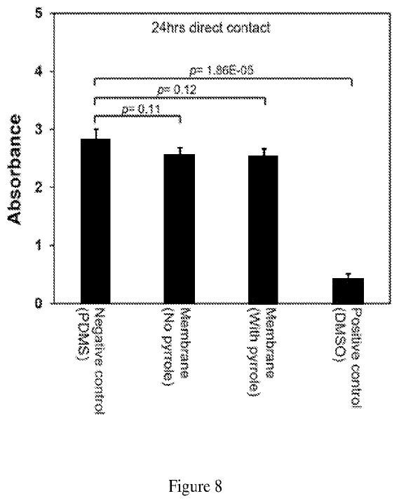

All components are dissolved in tetrahydrofuran (THF). The formation of a K⁺ sensor membrane is obtained by pipetting desired membrane solution volume on the commercial gold rod electrode surface modified with polypyrrole (PPy) and evaporating the THF solvent (overnight at room temperature). All experiments are performed in artificial interstitial fluid (ISF).
Preliminary results obtained with our recipe demonstrate exceptional selectivity when tested against 50 mM NaCl with high reproducibility. FIG. 1 (Na⁺ interference study. EMF response to 1 mM KCl and 50 mM NaCl additions) shows the response of a sensor to the addition of 1 mM KCl and 50 mM NaCl. The decreased KCl response is due to the logarithmic relation of concentration and electromagnetic field (EMF) signal.
FIG. 2 (Calibration plot. EMF response of the sensor to KCl additions (left), and respective calibration plot as logarithmic value of K⁺ concentration versus EMF change (right)) shows the calibration plot obtained using 10 wt % KI-II, 1.5 wt % CE, 29.5 wt % DOS, and 59 wt % PU. A log (K⁺) vs EMF linear range was obtained within 0.5 to 10 mM KCl.
FIGS. 3 and 4 show the results of the optimum CE and KI-II wt % studies when considering the sensor's selectivity to K⁺. The sensor was tested in artificial ISF against interferences from 2 mM Li⁺, 2 mM Mg⁺⁺, 4 mM Ca⁺, and 50 mM Na. The best response was observed when 15 wt % of KI-II and 3 wt % of CE, i.e. a 5 to 1 KI-II to CE ratio, was used. FIG. 5 shows the EMF curve obtained when using a membrane with this ratio. No significant overall EMF change was observed after adding any of the tested interfering ions at concentrations of their ISF counterparts.
FIG. 3—Optimization of cation exchanger concentration. Comparison of the potentiometric response of the sensor to selected interfering ions with respect to the response obtained to 1 mM KCl addition. Error bars represent the standard deviation of three independent experiments. Best results obtained with 1.5 wt % CE, 10/1.5 ratio of KI-II/CE.
FIG. 4—Optimization of K⁺ ionophore concentration. Comparison of the potentiometric response of the sensor to selected interfering ions with respect to the response obtained to 1 mM KCl addition. Error bars represent the standard deviation of three independent experiments. Best results obtained with 15 wt % KI-II, 15/3 ratio of KI-II/CE.
FIG. 5: Selectivity study. EMF response of a sensor to the addition of selected interference ions. Membrane recipe contains 15 wt % KI-II, 3 wt % CE, 27.3 wt % DOS, and 54.6 wt % PU.
PPy was used as an exemplary transducer layer throughout the experiments, but others might be used with expected similar results. The electrodeposition of PPy using chronoamperometry with +1 V continuous applied potential is shown in FIG. 6. The conductivity increase caused by PPy was analyzed by CV in ferro/ferricyanide redox probe and can be seen in the inset of FIG. 6. This PPy recipe resulted in desirable electrical conductivity and stability when the membrane was dissolved using tetrahydrofuran (THF) organic solvent and deposited onto the PPy-coated electrode. Conversely, CV-based PPy film lifted off after the membrane deposition step because of the THF solvent.
FIG. 6: Electropolymerization of polypyrrole. Chronoamperometric monitoring of polypyrrole deposition at 1 V applied potential for 1 min. The inset shows a comparison of the CV plots of a bare electrode with PPy-deposited electrode in 5 mM potassium ferro/ferricyanide redox couple solution.
FIG. 7: Preliminary PU/DOS wt % optimization study. EMF signal obtained from a sensor membrane containing different PU/DOS wt % ratios, and 10 wt % KI-II and 3 wt % CE.
Preliminary cytotoxicity study of the membrane with and without PPy is shown in FIG. 8. The cell viability assay was conducted using mouse fibroblast GPE 86 cell line seeded ˜5000 cells/well in a 96-well plate. A polydimethylsiloxane (PDMS) sample known to have no cytotoxicity was used as a negative control and dimethyl sulfoxide (DMSO) known to have cytotoxicity was used as a positive control. The sample specimens were placed directly on top of the cells after 24 hrs incubation (n=5) in the plate and cultured for 24 hrs at 37° C., 5% CO₂. The specimens were removed prior to adding the cell viability assay reagent. A colorimetric assay (cell counting kit 8, CCK-8) was used to quantify the metabolic activity of the viable cells. The morphologies of the cells were observed using an optical microscope after the exposure to the membrane specimens to compare the confluency of the cells after incubating the cells with the colorimetric assay reagents for 3 hrs. Cellular confluency observed with both membrane and PPy containing membrane samples is insignificantly different from that of negative control group. This indicates that the tested membrane samples do not induce cytotoxicity in the tested cells.
FIG. 8: Preliminary cytotoxicity study. Cell viability assay of membrane components with and without pyrrole. GPE 86 mouse fibroblast cell line was used to test direct contact of the membrane specimens. Error bars represent the standard deviation of 5 replicates.

### Example Discussion

Without being bound by a particular mechanism of action the following discussion of a preferred membrane composition is provided. The above-recited example of a preferred membrane composition shows superior sensor response and calibration curve, with a linear range covering the physiologically relevant range of K⁺ concentration in blood and interstitial fluid, including the clinically relevant areas of Hypokalemia, Normokalemia and Hyperkalemia. This membrane exhibits unprecedented selectivity for K⁺ detection at concentrations physiologically relevant in blood and interstitial fluid, when compared against equally relevant concentrations of other ions present in these human fluids, being the most prominent one Na⁺ (FIG. 1) but being equally selective against others such as Li⁺, Mg⁺⁺, and Ca⁺ (FIG. 5). Selectivity is proportional to the KI-II/CE wt % ratio. However, sensitivity, being another important parameter of a sensor, is just the opposite, which is defined as highest sensitivity with least interference, is 15/2.25, KI-II/CE wt % (FIGS. 3 and 4).

Sensitivity can be adjusted by modifying the PU/DOS wt % ratio, as it has been found to be inversely proportional to it. However, membrane integrity is also proportional to the PU/DOS wt % ratio (FIG. 7). DOS is important for K⁺ transport into the membrane from the ISF however, increased wt % can cause leakage and thus decreased signal reproducibility, membrane integrity, and membrane substrate adhesion. Similarly, as seen in FIG. 7, the selectivity of the sensor against 50 mM NaCl (being the largest interfering substance, as seen in FIG. 5) is directly proportional to the PU/DOS wt % ratio due to the increased EMF signal (relative to the EMF signal obtained to 1 mM KCl). PU/DOS wt % lower than 1/2 results in poor membrane integrity and adhesion to the electrode and therefore, lower ratios cause the EMF signal to deteriorate.

It has herein been discovered that the described ratios between KI-II/CE wt % and PU/DOS wt % produce a robust sensor membrane having superior response and performance for applications such as direct detection and determination of changes of K⁺ concentration in blood and/or interstitial fluid with an extremely high degree of confidence (low interference from other potential analytes), sensitivity, and resolution.

Furthermore, the membrane composition, as described herein, demonstrates that no cytotoxic reaction is observed when directly exposed to cells for a period of at least 24 hrs (FIG. 8), making it suitable for use on biomedical applications, where otherwise direct contact of the membrane with a patient tissue might result in irritation, discomfort and other potentially adverse reactions.

1. A functional K⁺ sensor membrane composition for use with a K⁺ specific sensor, the membrane composition comprising:
a K⁺ ionophore comprising (KI-II), a cation exchanger (CE) comprising (potassium tetrakis(4-chlorophenyl) borate), base membrane polymer (polyurethane (PU)), and plasticiser (dioctyl sebacate (DOS)); wherein
the composition has a wt %, mol % ratio of KI-II/CE of: 0-30.0 wt %.

2. The composition of claim 1, wherein the composition has a wt %, mol % ratio of KI-II/CE of: 0.6-10.0 wt %, 0.97-14.5 mol %, (e.g. 2/3-30/3 wt %); or 3.3-6.7 wt %, 4.8-9.7 mol %, (e.g. 10/3-10/1.5 wt %).

3. The composition of claim 1, wherein the composition has a wt % ratio PU/DOS wt % of: 0-10 (e.g. 0/10-10/1 wt %).

4. The composition of claim 4, wherein the wherein the composition has a wt % ratio PU/DOS wt % of: 0.2-4 (e.g. 1/5-4/1 wt %); or 0.3-2.5 (e.g. 1/3-5/2 wt %).

5. A K⁺ specific sensor comprising a functional K⁺ specific membrane, wherein the K⁺ specific membrane is formed from a functional K⁺ specific membrane composition of claim 1.

6. The sensor of claim 5, wherein the sensor is suitable for use in biological and biomedical applications, for example in connection with potassium sensors for determining potassium in biological fluids, such as blood and/or interstitial fluid), for example wherein the sensor is suitable for use in biological samples such as blood and/or interstitial fluid and for determining potassium levels in a linear range of 0.5 to 10 mM K⁺.

7. A functional K⁺ sensor membrane composition for use with a K⁺ specific sensor, the membrane composition comprising, consisting of, or consisting essentially of:
a K⁺ ionophore comprising (KI-II), a cation exchanger (CE) comprising (potassium tetrakis(4-chlorophenyl) borate), base membrane polymer (polyurethane), and plasticiser (dioctyl sebacate); wherein:
the composition has a wt % ratio PU/DOS wt % of: 0-10 (e.g. 0/10-10/1 wt %).

8. The composition of claim 7, wherein the composition has a wt % ratio PU/DOS wt % of: 0.2-4 (e.g. 1/5-4/1 wt %); or 0.3-2.5 (e.g. 1/3-5/2 wt %).

9. A K⁺ specific sensor comprising a functional K⁺ specific membrane, wherein the K⁺ specific membrane is formed from a functional K⁺ specific membrane composition of claim 7.

10. The sensor of claim 9, wherein the sensor is suitable for use in biological and biomedical applications, for example in connection with potassium sensors for determining potassium in biological fluids, such as blood and/or interstitial fluid), for example wherein the sensor is suitable for use in biological samples such as blood and/or interstitial fluid and for determining potassium levels in a linear range of 0.5 to 10 mM K⁺.

11. A functional K⁺ sensor membrane composition for use with a K⁺ specific sensor, the membrane composition comprising, consisting of, or consisting essentially of:
a K⁺ ionophore comprising (KI-II), a cation exchanger comprising (potassium tetrakis(4-chlorophenyl) borate), base membrane polymer (polyurethane), and plasticiser (dioctyl sebacate); wherein the components are present in the following amounts: K· ionophore (KI-II): 15 wt % (ionophore II); Cation exchanger (CE): 2.25 wt % (potassium tetrakis(4-chlorophenyl) borate); Plasticiser (DOS): 29.5 wt % (Bis(2-ethylhexyl) sebacate); and Polymer (PU): 58 wt % (polyurethane).

12. A K⁺ specific sensor comprising a functional K⁺ specific membrane, wherein the K⁺ specific membrane is formed from a functional K⁺ specific membrane composition of claim 11.

13. The sensor of claim 12, wherein the sensor is suitable for use in biological and biomedical applications, for example in connection with potassium sensors for determining potassium in biological fluids, such as blood and/or interstitial fluid), for example wherein the sensor is suitable for use in biological samples such as blood and/or interstitial fluid and for determining potassium levels in a linear range of 0.5 to 10 mM K⁺.

14. A method of forming a K⁺ specific sensor having a functional K⁺ specific membrane, the method comprising the steps of: (1) dissolving a K⁺ ionophore comprising (KI-II), a cation exchanger (CE) comprising (potassium tetrakis(4-chlorophenyl) borate), base membrane polymer (polyurethane (PU)), and plasticiser (dioctyl sebacate (DOS)) in a solvent to form a functional K⁺ sensor membrane composition, wherein the composition has a wt %, mol % ratio of KI-II/CE of: 0-30.0 wt %; and/or the composition has a wt % ratio PU/DOS wt % of: 0-10 (e.g. 0/10-10/1 wt %); and (2) applying the composition to a surface of an electrode and then evaporating solvent, thereby forming a K⁺ specific sensor having a functional K⁺ specific membrane.

15. The method of claim 14, wherein the electrode comprises gold (Au).

16. The method of claim 15, wherein the surface of the electrode is modified with polypyrrole (PPy).
